(19) United States
(12) Patent Application Publication
(10) Pub. No.: US 2012/0203248 A1
(43) Pub. Date: Aug. 9, 2012

(54) FOUR PRONGED ADJUSTABLE LEVER-ACTION CASTRATION TOOL

(76) Inventors: SCOTT WADSWORTH, ST. IGNATIUS, MT (US); MIKE WADSWORTH, ST. IGNATIUS, MT (US)

(21) Appl. No.: 13/020,344

(22) Filed: Feb. 3, 2011

(51) Int. Cl. A61B 17/10

(52) U.S. Cl. 606/141

(57) ABSTRACT

An easily operated castrating tool that allows placement of a continuous elastic band for ligation and removal of an appendage. The instant invention is also an improved adjustable spreading tool that allows for quick and easy placement of the improved elastic band about a body part to be ligated.

### BACKGROUND OF INVENTION

This invention generally relates to ligation type castration tools, and more particularly to an adjustable lever activated stretching tool for placing a continuous elastomeric band on animals for castration or removal of other body parts.

Background of the Invention. In animal husbandry, it becomes necessary at times to remove body parts and appendages. Examples of such actions include removing an animal's tail, testes, or other body parts. One way that this is done is to place a ligating band at the location where the body part is to be removed. The ligating band exerts sufficient tension upon the body part so as to restrict the flow of blood into and out of the body part. As a result of this restricted blood flow, the tissue within the appendage dies and after a period of time the appendage sloughs off.

Castration by ligation has become popular because of the simplicity of the process and the benefits it provides in avoiding undesirable consequences such as microbial infection, insect invasion, excessive bleeding, and the like. Ligation type castration of younger animals has been accomplished largely by use of small preformed continuous elastic or elastomeric bands, because of the economic viability provided by allowing rapid castration processing that can be accomplished by relatively unskilled persons.

The small elastic bands are generally placed by a spreading type tool having two arms interconnected in their medial portions to allow expansion of a band carried at one end portion of the tool against its elastic bias for placement over the scrotal pouch of the animal with subsequent release. This process works well with juvenile animals that do not have a mature testicular structure of larger size, but it has not been viable with larger animals having mature testicular structure such as bovine bulls, as a small preformed elastic band that would contract to a small enough configuration to provide ligation generally cannot be expanded sufficiently, either by its nature or by use of common spreading type tools, to allow passage over the larger testicular structure of the mature animal.

In addition, most devices for spreading the elastic ligation bands have not provided the user with the ability to sufficiently spread the elastic band so as to allow the passage of testes therethrough. Therefore, the use of elastic bands for ligation has generally been limited by the hand strength of the person utilizing the device.

Ligation castration with continuous preformed bands is easier and less complex than the formation of ligation banding in place. It has fewer possibilities for mistakes or errors. It provides a process that requires less care and skill on the part of an operator, and generally may be more easily accomplished by unskilled workmen without historical familiarity with the process. Elongate type ligation material that is formed in place also allows parameters for tensioning and band fastening by a clip that may be varied, either accidentally or deliberately by an operator, to provide results that are not necessarily uniform or consistent and may vary sufficiently to make the process inoperative or harmful to an animal.

Continuous band ligation material has more fixed parameters determined by the nature and configuration of the banding material itself, which are more independent of an operator's activities. The continuous banding material also is generally more durable than the elongate ligation material form in place, and is less expensive and more easily handled than the elongate material. There are therefore various advantages in using preformed continuous band type elastic material for ligation castrating, when the use of such material is possible.

The nature of the ligation castration process with small preformed continuous band elastic material defines the limits of the parameters required for the elastic bands usable for such purpose, and especially their relaxed size and elastic properties. Such bands must be small enough to fulfill their ligation purpose of providing sufficient elastic force or bias after placement and fastening about the neck of the scrotal pouch to cause atrophy of the tissue outwardly of the band while yet allowing sufficient expansion upon stretching to permit placement over the scrotal pouch.

Since a continuous ligation band must allow passage of the scrotal pouch and contain the testicular structure of an animal to be castrated through the orifice it defines and because of its small size, the bands must be enlarged by stretching to allow placement. The Applicant's invention found in U.S. Pat. No. 7,371,241, the disclosure of which is hereby incorporated by reference, provides a tool to simply and easily accomplish the stretching of such bands of the required nature to a size and configuration that allows placement without damage to the elastic material. U.S. Pat. No. 7,371,242 describes a system made up of a continuous elastomeric band that provides a desired amount of tensioning power together with a lever action band spreading tool that provides sufficient mechanical advantage to allow the bands in the present system to be sufficiently stretched so as to allow the bands to be placed over the appendages to be removed.

The bands typically used with the present invention are generally cylindrical bands with a flat top and a flat bottom. The walls of the cylinder have a thickness of about 5/16 of an inch when the rubber band is in the relaxed contracted state. These walls also have a height of about 5/16 of an inch when relaxed and an overall diameter of 11/16 of an inch. The bands define an inner opening having a circumference of about ¼ inch. Depending upon the differing requirements of use, the dimensions may be modified; however, the overall ratios of the size of the bands should be generally maintained. Bands having these ratios require a significant amount of force to open to a size sufficient to allow an appendage or body part, such as a scrotal pouch, to be inserted within the opening.

### SUMMARY OF INVENTION

The instant invention provides an easily operated adjustable castrating tool that allows placement of a continuous elastic band for ligation and removal of an appendage. The system of the present invention provides a device with sufficient stretching power so as to allow the bands of the present invention to be opened and for an appendage to be inserted therein and includes a mechanism to adjust the circumference the device will stretch a ligation band to for placement on objects of varying sizes.

The stretching tool of the present invention is a device made up of a pair of jaws, each having a portion configured to engage a portion of the ligating band. These jaws are also pivotally connected to a base portion, which is configured both for pivotal engagement with the jaws and for slideable engagement along a holding rail. The holding rail has a first end with a portion configured to connect and hold a pair of spacer bars and extends along a length to a handle portion which is configured to allow a user to hold and maintain the stretching tool in a desired position. The base plate is also connected to a lever. The lever has a first end pivotally connected to the base plate and extends along a length to a handle. Along the length of the lever, a bracing bar is pivotally attached. This bracing bar extends from a first end which is pivotally connected to the lever, along a length to a second end which is pivotally connected to an anchor near the handle portion of the holding rail. A pair of spacer bars is pivotally connected to the holding rail near its second end. These spacer bars extend from their connection with the holding rail along a length to a pivotal connection with a portion of the jaws. The spacer bars have extensions that are configured to engage a portion of the elastic band. In a preferred embodiment, the spacer bars have prong extensions that are configured to engage a portion of the elastic band.

The device is used by placing the lever such that the device is in a closed position. In this closed position, the lever is oriented in a generally perpendicular position with regard to the holding rod. To reach this position, the lever pivots about the base plate and the bracing bar, causing the base plate to be moved along the holding rail toward the handle portion of the rail. As this occurs, the spacer bars pivot about their connection with the holding rail and the connection with the jaws and pull the ends of the jaws towards the spacer bar prongs which are configured to hold a ligating band while pushing the spacer bar prongs closed and toward the ends of the jaws. In this position, the band grasping portions of the jaws, and the band grasping portions of said spacer bars, are in sufficiently close proximity so as to allow a ligating band to be placed upon the device.

Once a ligating band has been placed upon the device, the device may be moved into an open position. This is done by a user grasping the handle portion of the holding rail with one hand and the lever portion of the device with the other hand, and bringing the lever toward the holding rail. As force is applied to the lever, the lever pivots about both the pivoting connection between the base plate and the lever, as well as the connection between the bracing bars and the lever. As the lever pivots about these two points, the base plate slides along the holding rail. As the base plate moves along the holding rail, the spacer bars pivot about the connections between the spacer bars and the base plate or articulating device, and the spacer bars and the jaws.

As these spacer bars pivot, the jaws pivot about their connections with the base plate and the ends of the jaws are forced open as well as the prongs attached to the spacer bars and/or spacer bar extensions. In as much as the ligating band is connected to these jaws, as the jaws open the ligating band is stretched and the band is prepared for placement over a body part to be ligated. The combination of distances and multiple pivot points provide significant mechanical advantage to the person utilizing the device, thus allowing the bands to be opened for use with decreased amounts of effort or strength required on the part of the party utilizing the device.

The anchor in a preferred embodiment is adjustable to be connected at two different locations so as to alter the circumference to which the prongs of the spacer bar extensions and the jaws will stretch a ligation band. By adjusting the anchor to a second location further from the jaws of the device than when the anchor is in a first position, the distance between the handle portion of the holding rail and the grasping portion of the lever when the jaws and spacer bars are in a closed position is decreased, thus decreasing the distance the lever forces the base plate to travel along the holding rail. Moving the anchor from the first location to the second location limits the distance the holding rail will travel away from the jaws and thus limits the maximum stretching circumference the jaws and the spacer bars will stretch a ligation band to.

Once the body part to be ligated is placed within the opening defined by the stretched band, the lever can be returned again toward the closed position and the elastic band will close about the body part to be ligated. The device can then be removed, returned to a first position, loaded with another ligating band, and used again.

In a preferred embodiment, the location of the pivoting connection between the holding rail and the articulating device is configured to be adjustable to at least two different positions at each of which the maximum stretching circumference of the band varies. When pivoting location is at a first position, the device is configured to stretch a resilient band to a first position maximum circumference. When the pivoting location is at a second position, the device is configured to stretch a resilient band to a second position maximum circumference. The first position maximum circumference and the second position maximum circumference are not equal.

In a preferred embodiment, the pivot area is configured to be slideable from the first location to the second location, where the maximum circumference to which the tool will stretch an endless loop is different than the maximum circumference to which the tool would stretch an endless look in the first position. The movement of the pivot location from the first location to the second location alters the positioning of the articulating device in order to alter the maximum circumference to which the tool will stretch an endless loop.

In a preferred embodiment, the holding bar has an opening near the first articulating device pivoting position into which a pin inserts to hold the articulating device at the first position. In a preferred embodiment, the holding bar has an opening near the second articulating device pivoting location into which a removable pin inserts to hold the articulating device at the second position. In a preferred embodiment, the pin(s) is releasably inserted into each of the opening near the first articulating device pivot location or the opening near the second articulating device pivoting position to releasably hold the articulating device in the first location or the second location.

This system provides an adjustable device for ligating body parts from animals that is significantly easier to use, and more simple and efficient as compared to other methods that exist in the prior art. In addition, this invention provides an adjustable device for placing ligating bands, which reduces the incidence of unintended injury to animals as compared to other devices and systems known in the art.

Still other objects and advantages of the present invention will become readily apparent to those skilled in this art from the following detailed description wherein I have shown and described only the preferred embodiment of the invention, simply by way of illustration of the best mode contemplated by carrying out my invention. As will be realized, the invention is capable of modification in various obvious respects all without departing from the invention. Accordingly, the drawings and description of the preferred embodiment are to be regarded as illustrative in nature, and not as restrictive.

### BRIEF DESCRIPTION OF DRAWINGS

FIG. 1 is a perspective top view of an embodiment of the present invention comprised of elastic ligating bands, and the tool for placement of these bands.

FIG. 2 is a perspective bottom view of an embodiment of the present invention in a first position.

FIG. 3 is a detailed top view of the tool shown in FIG. 2, showing placement of a ligation band thereupon.

FIG. 4 is a top perspective view of an embodiment of the invention in a first configuration showing a ligation band placed and stretched thereupon.

FIG. 5 is a top perspective view of an embodiment of the invention in a second configuration showing a ligation band placed and stretched thereupon.

### DESCRIPTION OF THE PREFERRED EMBODIMENT

While the invention is capable of various modifications and alternative constructions, certain illustrated embodiments thereof have been shown in the drawings and will be described below in detail. It should be understood, however, that there is not intention to limit the invention to the specific form disclosed, but, on the contrary, the invention is to cover all modifications, alternative constructions, and equivalents falling within the spirit and scope of the invention as defined in the claims.

The instant invention provides an easily operated adjustable castrating tool 10 that allows placement of a continuous elastic band 8 for ligation and removal of an appendage. The tool of the present invention provides a tool that allows such bands 8 to be placed in a manner that is easy to use, adjustable, and less fatiguing to the parties that utilize the device 10. The device has an adjustment mechanism that is configured to allow for varying the maximum stretching size that the device will stretch a ligation band in each configuration. The instant invention includes an elastic band 8 which allows for better compressive ligation about a body part as well as a simple, but effective, spreading tool 10 that allows for quicker and easier placement of the elastic band 8 about a body part to be ligated.

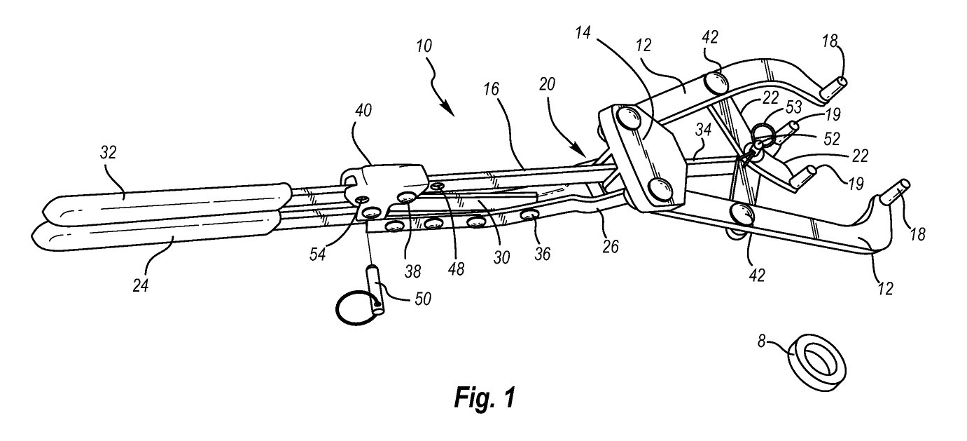

FIG. 1 shows a top perspective view of system of the present invention. The system of the present invention is made up of a tool 10 and a variety of bands 8 which are configured to be placed about a body part to be ligated.

The bands 8 of the present invention are generally cylindrical bands having a flat top and a flat bottom. In a preferred embodiment, the walls of the cylinder have a thickness of about 5/16 of an inch when the band is in the relaxed contracted state. These walls also have a height of about 5/16 of an inch when relaxed and an overall diameter of 11/16 of an inch. The bands define an inner opening having a circumference of about ¼ inch. Depending upon the differing requirements of use, the dimensions may be modified; however, the overall ratios of the size of the bands 8 should be generally maintained. These bands also have additional features in that they are coated with an antiseptic property, such as an antiseptic powder, which further fights against infection and other negative possible side effects from the ligation procedure.

Bands 8 having these ratios require a significant amount of force to open to a size sufficient to allow an appendage or body part, such as a scrotal pouch, to be inserted within the opening. The system of the present invention provides a device with sufficient stretching power so as to allow the bands 8 of the present invention to be opened and for an appendage to be inserted therein.

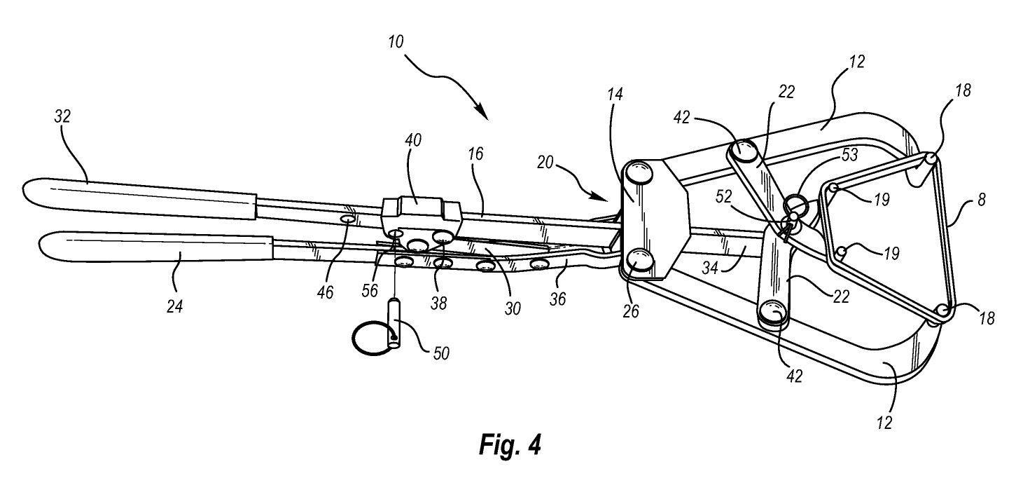

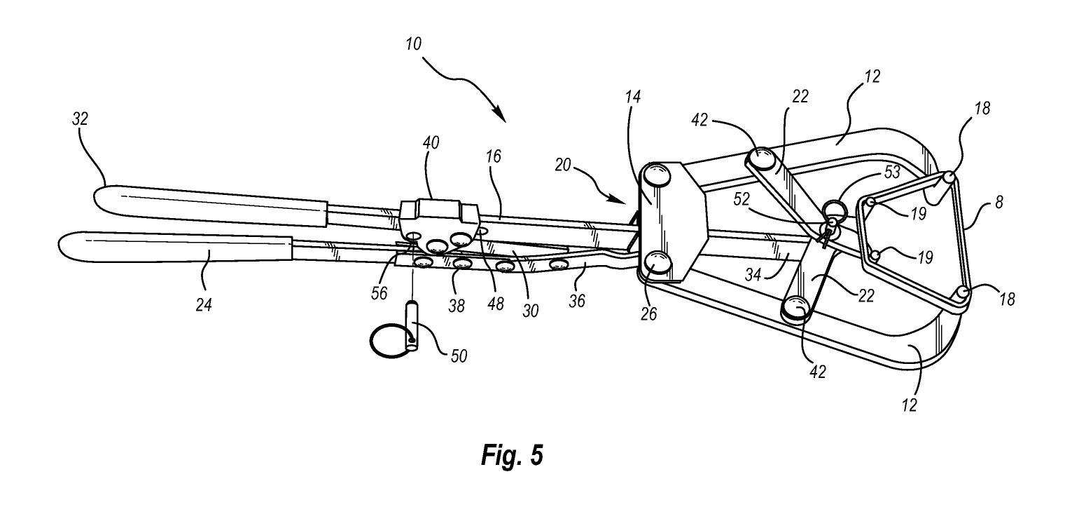

The stretching tool 10 of the present invention is a device made up of a pair of jaws 12, each having a portion 18 configured to engage a portion of the ligating band 8. These jaws 12 are also pivotally connected to a base portion 14, which is configured both for pivotable engagement with the jaws 12 and for slideable engagement along a holding rail 16. This base portion 14 is a part of an articulating device 20 which is utilized that is used in conjunction with a lever 24 to move the spacer bars 22 to spread the band 8. The holding rail 16 has a first end 34 with a portion 19 configured to connect the spacer bars 22 to the holding rail 16, and extends along a length to a handle portion 32 which is configured to allow a user to hold and maintain the stretching tool 10 in a desired position. The base plate 14 is also connected to a lever 24. The lever 24 has a first end 26, which is pivotally connected to the base plate 14 and extends along a length to a handle 32. Along the length of the lever 24 a bracing bar or arm 30 is pivotally attached. This bracing bar 30 extends from a first end 36, which is pivotally connected to the lever 24, along a length to a second end 38, which is pivotally connected to an anchor 40 positioned near the handle portion 32 of the holding rail 16. The anchor 40 location is configured to be slideable from a first position (shown in FIG. 1 and FIG. 4) on the holding rail to a second position (shown in FIG. 5) on the holding rail. In the depicted embodiment, the holding rail 16 has two holes 48 46 (shown in FIG. 4) into which pin 50 can be inserted through the opening 54 in the anchor 40 to hold the anchor 40 in place at either the first position or the second position on the holding arm 16. A pair of spacer bars 22 is pivotally connected to the holding rail 16 near its second end 34 by having prong 52 inserted through the spacer bars. In the depicted embodiment, the spacer bars are held on the prong 52 by a cotter pin 53 that is inserted through the pin 52. In a preferred embodiment, there is a washer (not shown) placed between the spacer bar and the opening in the spacer pin 42 that is positioned between the spacer bar and the cotter pin. In a preferred embodiment, each spacer bar 22 extends from its connection with the holding rail 16 along a length to a pivotal connection 42 with a portion of the jaws 12 and from its connection with the holding rail 16 to an end having a prong 19 that is configured to hold a band and stretch a band in conjunction with the other spacer bar prong 19 and the two jaw prongs 18.

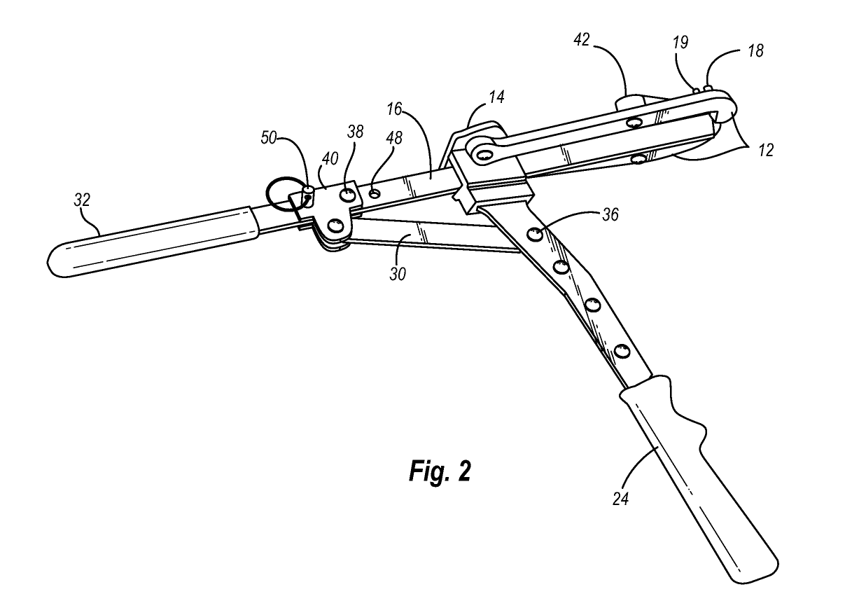

FIG. 2 shows the tool 10 of the present invention in a closed position. In this closed position, the lever 24 is oriented in a generally perpendicular position with regard to the holding rod 16. In this closed position, the lever 24 pivots about the base plate 14 and the bracing bar 30, causing the base plate 14 to be moved along the holding rail 16 toward the handle 32 portion of the rail 16. As this occurs, the spacer bars 22 pivot about their connection with the holding rail 16 and the connection with the jaws 12 and pull the ends of the jaws 18 towards the portion 19 of the spacer bars 22 which is configured to hold a ligating band 8 while pushing the prongs 10 on the spacer bars 22 toward the jaw 18. In this position, the band grasping portions of the jaws 18 and the spacer bars 19 are in sufficiently close proximity so as to allow a ligating band 8 to be placed upon the device 10.

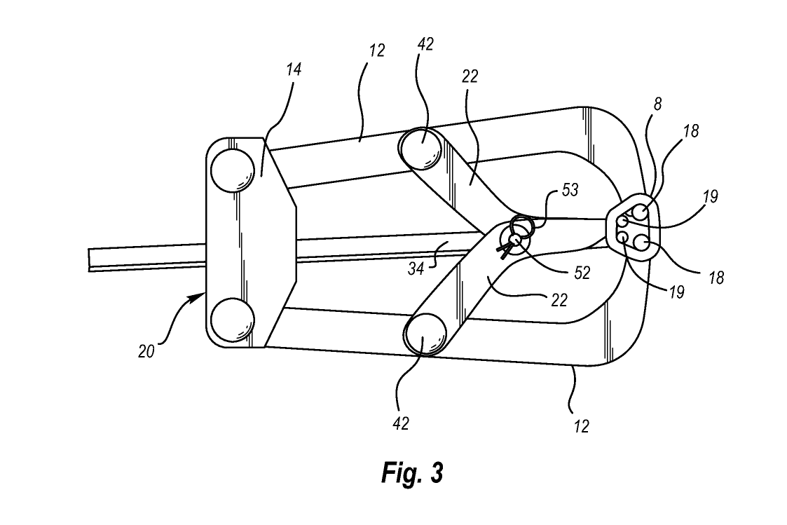

A detailed top view of the positioning of the jaws and the ligating band placed upon the tool is shown in FIG. 3.

While in FIG. 2 the position of the holding rail 16 and the lever 24 is described as being generally perpendicular, in the preferred embodiment the position of the lever 24 with regard to the holding rail 16 bar varies with which opening 46 48 the pin is inserted into. This feature provides a variety of benefits. First, this position assists in providing mechanical advantage sufficient to produce an initial force sufficient to stretch the band 8. By allowing a longer path of travel for the lever 24, the lever 24 is able to pivot about the connection between the bracing bar 30 and the lever 24. This creates a force which is then transferred and sent to the connection between the lever 24 and the base plate 14. The lengths of the spacing arms 22 and the pivots involved provide increases in force placed upon the band and allow the band 8 to stretch all without a significant amount of work on the part of the person utilizing the device 10.

An embodiment of the invention in a first open position with a stretched band is shown in FIG. 4.

Once a ligating band 8 has been placed upon the device 10, the device 10 may be moved into an open position. Moving the device 10 into the open position is done by a user grasping the handle 32 portion of the holding rail 16 with one hand and the lever 24 portion of the device with the other hand and bringing the lever 24 toward the holding rail 16. As force is applied to the lever 24, the lever 24 pivots about both the pivoting connection between the base plate 14 and the lever 24, as well as the connection between the bracing bar 30 and the lever 24. As the lever 24 pivots about these two points, the base plate 14 slides along the holding rail 16. As the base plate 14 moves along the holding rail 16, the spacer bars 22 pivot about the connections between the spacer bars 22 and the holding rail 16, and the spacer bars 22 and the jaws 12. As these spacer bars 22 pivot, the jaws 12 pivot about their connections with the base plate 14 and the ends of the jaws 12 are forced open and the ends of the spacer bars 19 are forced apart. In as much as the ligating band 8 is connected to these jaws 12 and spacer bars 19, as the jaws 12 open the ligating band 8 is stretched and the band 8 is prepared for placement over a body part to be ligated.

The combination of distances and multiple pivot points provide significant mechanical advantage to the person utilizing the device, this allowing the bands 8 to be opened for use with decreased amounts of effort or strength required on the part of the party utilizing the device. An additional advantage of the present invention is that the device 10 can be maintained in an open position with one hand.

In order to move the device into the open position, the lever 24 must be moved from the closed position shown in FIG. 2 toward the handle 32 that is positioned upon the holding rod 16 to the position shown in FIG. 4. At this position, the band 8 compresses the ends of the jaws 12 toward each other and the ends of the prongs 19 toward each other and pushes the four prongs toward the closed position. This compressive force causes the base plate 14 to be moved toward the first end 34 of the holding rod 16. This force then causes the lever 24 to pivot about the connection between the lever and the base plate 14 and the connection between the bracing arm 30 and the lever 24. This causes the lever 24 to be moved toward the handle 32 of the holding rod 16 thus placing the device in the first position shown in FIG. 1 and FIG. 4. The present invention can be configured to utilize the naturally compressive forces of the elastic band to hold the stretching device in an open position and allows a user to hold a stretched device in an open position with just one hand and frees up the other hand for use in other activities.

Once the body part to be ligated is placed within the opening defined by the stretched band 8, the lever 24 can be returned again toward the closed position, and the elastic band 8 will close about the body part to be ligated. The device can then be removed, returned to a closed position, loaded with another ligating band 8, and used again.

This system provides a method and device for ligating body parts from animals that is significantly easier to use, more simple and efficient as compared to other methods which exist in the prior art. In addition, this invention provides a system and method for placing ligating bands which reduces the incidence of unintended injury to animals as compared to other devices and systems known in the art.

While there is shown and described the present preferred embodiment of the invention, it is to be distinctly understood that this invention is not limited thereto but may be variously embodied to practice within the scope of the following claims. From the foregoing description, it will be apparent that various changes may be made without departing from the spirit and scope of the invention as defined by the following claims.

What is claimed is the following:

1. A device for stretching an endless loop of resiliently stretchable material for use in removing body parts by constricting blood flow, said device comprised of a holding rail;
a pair of jaws configured to engage a portion of said endless loop, said jaws further pivotally connected to a base portion, said base portion configured for slideable engagement along said holding rail in a first plane of movement, said holding rail having a portion connected to a grasping handle, and an articulating device configured to move said base portion along said holding rail so as to manipulate said jaws, so as to engage and stretch said endless loop, said articulating device including a pair of spacer bars pivotally connected to said jaws, and pivotally connected to a portion of said holding rail at an anchor location, wherein said spacer bars comprise at least portions configured to hold a portion of said endless loop, said spacer bars configured to alternatively open and close said jaws and said prongs on said spacer bars when said base portion is alternatively moved along said holding rail;
wherein said anchor location is configured to be adjustable to at least a first position and a second position, wherein said device is configured to stretch a resilient band to a first position maximum circumference when said device is in said first position, wherein said device is configured to stretch a resilient band to a second position maximum circumference when said device is in said second position, wherein said device is configured such that said first position maximum circumference is greater than said second position maximum circumference.

2. The device for stretching an endless loop of resiliently stretchable material of claim 1, wherein said articulating device is configured to be pivotally connected to an anchor at said anchor location, wherein said anchor is configured to be releasably connected to said holding rail at a first location, wherein said anchor is configured to be movable from said first location on said holding rail to a second location on said holding rail wherein said anchor is configured to be releasably attached to said holding rail.

3. The device of claim 2, wherein said holding rail comprises a first opening approximate to said first location and a second opening approximate to said second location, wherein said device comprises at least one pin configured to insert through said anchor and said first opening to releasably hold said anchor in said first location, wherein said device comprises at least one pin configured to insert through said anchor and said second opening to releasably hold said anchor in said second location.

4. A device for stretching an endless loop of resiliently stretchable material for use in removing body parts by constricting blood flow, said device comprised of a holding rail;
a pair of jaws configured to engage a portion of said endless loop, said jaws pivotally connected to a base portion, said base portion configured for slideable engagement along a holding rail in a first plane of movement, said holding rail having a portion connected to a grasping handle;
an articulating device configured to move said base portion along said holding rail so as to manipulate said jaws, so as to engage and stretch said endless loop, said articulating device moving in a second plane generally perpendicular to said first plane of movement;
wherein said holding rail is pivotally connected to a pair of spacer bars, said spacer bars also pivotally connected to said jaws, whereby advancing said base portion along said holding rail causes said jaws to open by moving said base portion toward said spacer bars causing said spacer bars to pivot at said connection with said holding rail and at said connections with said jaws;
wherein said spacer bar extensions are configured and positioned such that when said spacer bars pivotally open said jaws said spacer bars separate said spacer bar extensions, wherein said device comprises a mechanism to limit the width at which said spacer bars will space apart said jaws;
wherein each of said spacer bars comprises a spacer bar extension, wherein said spacer bar extensions comprise a portion configured to engage a portion of said endless loop;
wherein said device is configured to manipulate said spacer bar extensions so as to move said spacer bar extensions so as to engage and stretch said endless loop in combination with said jaws when said device manipulates said jaws.
